(19) United States
(12) Patent Application Publication
(10) Pub. No.: US 2006/0252026 A1
(43) Pub. Date: Nov. 9, 2006

(54) Cryopreservation method for bivalve oocytes

(76) Inventors: Harry Robin Tervit, Papamoa (NZ); Serean Leigh Adams, Nelson (NZ); Rodney David Roberts, Nelson (NZ); Lindsay Thomas McGowan, Hamilton (NZ); Patricia Anne Pugh, Hamilton (NZ); John Frederick Smith, Hamilton (NZ); Achim Ralph Janke, Nelson (NZ)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/377,684

(22) Filed: Mar. 17, 2006

(30) Foreign Application Priority Data
Mar. 17, 2005 (NZ) NZ 538902

(51) Int. Cl. C12N 5/06; A01N 1/02

(52) U.S. Cl. 435/2; 435/325

(57) ABSTRACT

A method for cryopreserving bivalve oocytes, particularly those of Pacific oysters, by preparing a solution of a cryoprotectant in water, combining this with the oocytes, carrying out a first cooling step to a temperature of about −4° C. to −20° C., and carrying out a second cooling step to a temperature of about −25°°C. to −55° C.

### TECHNICAL FIELD

The invention relates to a method for cryopreserving bivalve oocytes, in particular Pacific oyster (Crassostrea gigas) oocytes. The method includes the preparation of a cryoprotectant solution, combining the oocytes with the cryoprotectant solution, and cooling the resultant mixture in two distinct cooling steps.

### BACKGROUND

The bivalve shellfish industry is an important one, both in New Zealand and worldwide. For example, Pacific oysters are farmed throughout the world with an annual production of approximately 4,200,000 metric tonnes (FAO 2004). In many countries, juvenile spat are produced in hatcheries and then cultivated on farms to marketable size. The use of hatchery reared spat has provided opportunities for selective breeding programmes to establish superior genetic lines. Cryopreservation goes hand in hand with selective breeding as it enables hatcheries to have total control over parental crosses and reduces the cost of maintaining a large number of broodstock from different family lines. Cryopreservation also enables year-round spat (juvenile) production without the costs of having to mature broodstock out of season. The development of suitable cryopreservation techniques is therefore important for the aquaculture industry.

However, the aquaculture industry requires that thousands to hundreds of thousands of oocytes be successfully cryopreserved. This contrasts with the small number of oocytes used for successful cryopreservation in human IVF or in agriculture. Furthermore, although there has been progress in the development of techniques for cryopreservation of bivalve sperm and embryos, efforts to cryopreserve bivalve oocytes and successfully fertilise these and rear the larvae to spat and eventually to mature adult oysters have not to date, been successful.

Cryopreservation of Pacific oyster sperm has been reported by a number of groups (e.g.: S Ieropoli, P Masullo, M Do Espirito Santo, G Sansone, Cryobiology (2004) 49, 250; Q Dong, B Eudecline, C Huang, S K Allen, T R Tiersch, Cryobiology (2005) 50, 1). The applicant has also developed a commercially viable method (S L Adams, J F Smith, R D Roberts, A R Janke, H F Kaspar, H R Tervit, P A Pugh, S C Webb, N G King, Aquaculture (2004) 242, 271).

Methods for cryopreserving oyster larvae have also been investigated by a number of groups.

According to one such method, Lin et al. were able to obtain up to about 80% survival rates for cryopreserved oyster embryos using either dimethyl suffoxide (Me₂SO) or glycerol as cryoprotectants (CPA), and a two-step freezing procedure (Lin, N-H Chao, H-T Tung, Cryobiology (1999) 39, 192).

Gwo investigated some of the important variables for successful cryopreservation of oyster embryos (J-C Gwo, Theriogenology (1995) 43, 1163). Best results were obtained with trochophore embryos which were pre-equilibrated with propylene glycol (PG) and then cooled at a rate of 2.5° C. min⁻¹ to −30° C.

Paniagua-Chavez el al. have shown that cryopreserved Eastern Oyster (Crassostrea virginica) trochophore larvae can develop post-thaw to spat and eventually to adult oysters (C G Paniagua-Chavez, J T Buchanan, J E Supan, T R Tiersch, Cryo-Letters, 19, 283). The cryopreservation method used a freezing diluent solution composed of artificial seawater and PG as the CPA.

However, in contrast to the moderate success achieved in cryopreserving bivalve larvae and sperm, the cryopreservation of bivalve oocytes has, to date, been extremely difficult. Only three studies have described attempts to cryopreserve Pacific oyster oocytes. However, post-thaw development, where reported, was exceptionally low.

Naidenko attempted the cryopreservation of Pacific oyster oocytes using Me₂SO as CPA. In the presence of trehalose (T Naidenko, Cryo-letters, (1997) 18, 375). Freezing was carried out in three steps, to a final temperature of −35° C. However, the results of post-thaw development were extremely poor, with less than 0.5% fertilisation achieved with the thawed oocytes and less than 0.005% developing to D-larvae.

Chen et al. have also reported efforts to cryopreserve Pacific oyster oocytes (C-P Chen, H-W Hsu, S-F Lei and C-H Chang, J. Fish Soc. Taiwan, (1989) 16(3), 197). The procedure of Chen et al. involved a preincubation of the oocytes with Me₂SO, then freezing in two stages to a temperature of −30° C., followed by a more rapid freezing step to −150° C. and then storage in liquid nitrogen. However, the authors do not report fertilisation or development following the introduction of sperm to thawed egg suspensions.

Staeger briefly reported an attempt to cryopreserve Pacific oyster oocytes using Me₂SO as CPA. However, these efforts were entirely unsuccessful (W H Staeger, MSc Thesis, Oregon State University 1973). Paniagua-Chavez reported the attempted cryopreservation of Eastern Oyster (Crassostrea virginica) oocytes, using Me₂SO or sucrose as CPA. However, these efforts resulted in only 7% of the oocytes staining as live after cryopreservation, and none of the oocytes were able to be fertilised (C G Paniagua-Chavez, PhD Thesis, Louisiana State University, 1999).

There is therefore a need for a successful method for cryopreserving oocytes of Pacific oyster and other bivalves. Such a method will be useful in selective breeding programmes and hatcheries, as well as for genetically standardised bioassay work.

It is an object of the invention to provide a method for cryopreserving bivalve oocytes, or at least to provide a useful choice.

### STATEMENTS OF INVENTION

In a first aspect the invention provides a method of cryopreserving bivalve shellfish oocytes, including the steps of:

(a) preparing a solution comprising 5-50% v/v of a cryoprotectant in water to give a cryoprotectant solution;

(b) combining the oocytes with the cryoprotectant solution to give an oocyte mixture;

(c) carrying out a first cooling of the oocyte mixture at a rate of about 0.1° C. min⁻¹ to 20° C. min⁻¹ to a temperature of about −4° C. to −20° C.; and

(d) carrying out a second cooling of the oocyte mixture at a rate of about 0.1° C. min⁻¹ to 2.5° C. min⁻¹ to a temperature of about −25° C. to −55° C.

The cryoprotectant may be a permeating cryoprotectant or a non-permeating cryoprotectant The permeating cryoprotectant may be selected from, but is not limited to, the group consisting of dimethyl sulfoxide, ethylene glycol, and propylene glycol, and is preferably dimethyl sulfoxide or ethylene glycol. The non-permeating cryoprotectant may be selected from, but is not limited to, the group consisting of trehalose, sucrose, and arabinogalactan.

It is further preferred that the cryoprotectant solution in step (a) also contains about 0-10% v/v of a polyvinylpyrrolidone.

The cryoprotectant solution in step (a) is preferably prepared using seawater or purified water, ideally purified water.

It is also preferred that prior to step (b) the oocytes are suspended in or combined with seawater.

It is preferred that prior to step (b) the oocytes are suspended in or combined with seawater, preferably at a density of about 1600 to 2,000,000 oocytes mL⁻¹.

The first cooling of the oocyte mixture in step (c) may be carried out at any rate in the range of about 0.1° C. min⁻¹ to 20° C. min⁻¹. However, the rate is preferably about 0.1° C. min⁻¹ to 10° C. min⁻¹, more preferably about 0.1° C. min⁻¹ to 5° C. min⁻¹, and typically about 1° C. min⁻¹.

The second cooling of the oocyte mixture in step (d) may be carried out at any rate in the range of about 0.1° C. min⁻¹ to 2.5° C. min⁻¹. However, the rate is preferably about 0.1° C. min⁻¹ to 2.0° C. min⁻¹, more preferably about 0.1° C. min⁻¹ to 1.5° C. min⁻¹, even more preferably about 0.1° C. min⁻¹ to 1.0° C. min⁻¹, most preferably about 0.1° C. min⁻¹ to 0.5° C. min⁻¹, and typically about 0.3° C. min⁻¹.

The combining of the oocytes with the cryoprotectant solution in step (b) may be carried out in 1 to 20 steps, optionally with mixing between each step. It is preferred that there is a time interval between each step of about 1 minute. Alternatively, the combining of the oocytes with the cryoprotectant solution in step (b) may be carried out in a single step.

Preferably the cooling of the oocyte mixture in step (c) is carried out to a temperature of about −10° C. to −16° C. Furthermore, it is preferred that the cooling of the oocyte mixture in step (d) is carried out to a temperature of about −25° C. to −45° C., more preferably about −35° C. to −45° C.

It is preferred that the method includes a further step (e) between steps (b) and (c):

(e) loading the oocyte mixture into one or more vessels suitable for freezing.

The vessels are preferably straws or cryovials.

The method may also include a step (f) between steps (c) and (d):

(f) checking whether ice crystals have formed in step (c), and, if no ice crystals have formed, manually seeding the oocyte mixture to induce ice formation.

The method may further include a step (g) between steps (c) and (d):

(g) holding the oocyte mixture at a temperature of about −4° C. to −20° C. for up to 15 minutes.

Preferably the oocyte mixture in step (g) is held at the temperature for about 5 minutes.

Preferably the solution in step (a) comprises about 10-40% v/v of the cryoprotectant, most preferably about 20-30% v/v. Furthermore, it is preferred that the solution in step (a) comprises about 0.34-8.5% w/v of a polyvinylpyrrolidone, most preferably about 1.7% w/v. The polyvinylpyrrolidone may be of any molecular weight.

In a particularly preferred embodiment, the concentration of the cryoprotectant in the oocyte mixture after step (b) is about 10-15% v/v. In another preferred embodiment, the concentration of the polyvinylpyrrolidone in the oocyte mixture after step (b) is about 0.85% w/v.

The method may also include a step (h) between steps (b) and (c):

(h) holding the oocyte mixture at a temperature of about −3° C. to 30° C. for up to 20 minutes.

Preferably the oocyte mixture in step (h) is held at a temperature of about 15° C. to 30° C. It is also preferred that the oocyte mixture in step (h) is held at that temperature for about 5 to 20 minutes. The mixture may then be held at a temperature of 0° C. to 6° C. for approximately 5 minutes.

The method may include a step (i) after step (d):

(l) holding the oocyte mixture at a temperature of about −25° C. to −55° C., for up to about 20 minutes.

The method may also include a step (j) after step (i) or step (d):

(j) storing the oocyte mixture at a temperature below about −130° C., preferably in liquid nitrogen.

After storage in liquid nitrogen for any desired time period, the method may include a step (k):

(k) thawing the oocyte mixture at about 20° C. to 30° C.

The bivalve shellfish oocytes used in the invention are preferably oocytes of any one of the genera selected from the group consisting of Crassostrea, Saccostrea, Perna, Mytilus, Tapes, Ruditapes, Mercenaria, Panopea, Pinctada, Tridacna, Anadara, Ostrea, Pecten, Argopecten, and Tiostrea.

Preferably the bivalve shellfish oocytes are oocytes of any one of the species selected from the group consisting of Crassostrea ariakensis, Crassostrea virginica, Crassostrea sikarnea, Saccostrea glomerata (commercialis), Saccostrea cucullata, Perna viridis, Perna canaliculus, Perna perna, Mytilus edulis, Mytilus galloprovincialis, Mytilus trossulus, Ruditapes philippinarum, Ruditapes variegata, Ruditapes decussates, Ruditapes largillierti, Mercenaria mercenaria, Pecten yessoensis, Pecten maximus, Pecten fumatus, Argopecten irradians, Pecten novaezelandiae, Panopea abrupta, Panopea zelandica, Pinctada margaritifera, Pinctada maxima, Pinctada fucata, Tridacna gigas, Tridacna maxima, Tridacna squamosa, Tridacna derasa, Anadara granosa, Ostrea edulis, Ostrea chilensis, Ostrea angasi, and Austrovenus stutchburyl.

It is further preferred that the bivalve shellfish oocytes are oocytes of Crassostrea gigas (Pacific oyster).

### BRIEF DESCRIPTION OF THE FIGURES

FIG. 1 shows the effect of cryoprotectant (CPA) type and CPA concentration (% v/v) on percentage oocyte fertilisation post-thaw (mean±se, n=3): ethylene glycol [EG]; dimethyl sulfoxide [Me₂SO]; propylene glycol [PG] (Example 2.1).

FIG. 2 shows effect of addition of the extracellular CPAS, polyvinylpyrrolidone—PVP-40 and/or trehalose (T), to base CPA solution (10% EG in MQ) on percent oocyte fertilisation post-thaw (mean±se, n=3) (Example 2.2).

FIG. 3 is an illustration of main effects on percentage oocyte fertilisation post-thaw (mean±se, n=12) from combined analysis of Examples 2.3 and 2.4. CPA type: ethylene glycol [EG]; dimethyl sulfoxide [Me₂SO]; water type: Milli-Q® water (MO); sea water [SW]; extracellular CPA: polyvinylpyrrolidone—PVP-40 [+PVP]; no PVP (Examples 2.3 and 2.4).

FIG. 4 shows the effect of the isothermic holding temperature on percentage oocyte fertilisation post-thaw (mean±se, n=3). None=no hold versus hold at specified temperature for 5 minutes (Example 2.5).

FIG. 5 shows the effect of freezing rate post isothermic hold on percentage oocyte fertilisation post-thaw (mean±se, n=3) (Example 2.6)

### DETAILED DESCRIPTION

This invention is the first successful method for cryopreservation of bivalve oocytes.

Advantageously, the method of the invention enables high proportions of the frozen oocytes, once thawed, to fertilise and develop normally to eyed larvae and spat. To date, efforts to rear bivalves from cryopreserved eggs have only given very poor results (see, for example, F Hamarato\check{g}lu, A Ero\check{g}lu, M Toner, K C Sadler, Cryobiology (2005) 50, 38; T Naldenko, Cryo-letters (1997) 18, 375). In fact, oocyte cryopreservation is not routine for any species and the number of oocytes that can be cryopreserved using the present method is quite unprecedented. Furthermore, the method of the invention is the first successful method for the cryopreservation of high numbers of oocytes of any aquatic species.

Importantly, it is possible, using the present method, to produce the numbers of larvae required for selective breeding. In addition, the method allows smaller numbers of oocytes to be frozen, with a view to using these in genetically standardised or out-of-season bioassay work. The method of the invention can also be used to enable top quality larvae to be produced at any time of year for aquaculture.

Pacific oyster (Crassostrea gigas) oocytes can be successfully cryopreserved using the method of the invention. It will be dear to the skilled person that the method can equally be applied to the freezing of oocytes of other bivalve species such as those of the genera Crassostrea, Saccostrea, Perna, Mytilus, Tapes, Ruditapes, Mercenaria, Panopea, Pinctada, Tridacna, Anadara, Ostrea, Tiostrea, Pecten, and Argopecten. The bivalve species include Crassostrea ariakensis, Crassostrea virginica, Crassostrea sikarnea, Saccostrea glomerata (commercialis), Saccostrea cucullata, Perna viridis, Perna canaliculus, Perna perna, Mytilus edulis, Mytilus galloprovincialis, Mytilus trossulus, Ruditapes philippinarum, Ruditapes variegata, Ruditapes decussates, Ruditapes largillierti, Mercenaria mercenaria, Pecten yessoensis, Pecten maximus, Pecten fumatus, Argopecten irradians, Pecten novaezelandiae, Panopee abrupta, Panopea zelandica, Pinctada margaritifera, Pinctada maxima, Pinctada fucata, Tridacna gigas, Tridacna maxims, Tridacna squamosa, Tridacna derasa, Anadara granosa, Ostrea edulis, Ostrea chilensis, Ostrea angasi, and Austrovenus stutchburyi.

The method is also suitable for freezing oocytes over a wide range of densities. As noted above, one of the advantages of the present method is that it can be used to freeze oocytes at densities greater than 1600 eggs mL⁻¹. and even up to two million eggs mL⁻¹.

The invention uses a CPA, which may be a permeating CPA or a non-permeating CPA. A permeating CPA is a CPA that can traverse a cell plasma membrane. It will be clear to a person skilled in the art that any suitable permeating CPA can be used. However, it is preferred that the CPA used is ethylene glycol (EG) or Me₂SO, preferably at 10% v/v and 15% v/v respectively. It is most preferable to use 10% EG as the CPA, partly because it involves fewer handling issues than Me₂SO. The toxicity of CPAs is dependent on type and concentration, the equilibration time, the temperature during loading and the developmental stage of the organism being cryopreserved, and the skilled person will select a suitable CPA on these bases (see J-C Gwo, Cryopreservation of Eggs and Embryos from Aquatic Organisms in “Cryopreservation in Aquatic Species”, Eds. T R Tiersch and M P Mazik, World Aquaculture Society, Baton Rouge, 2000). Any suitable non-permeating CPA may also be used in the invention. Examples include trehalose, sucrose, and arabinogalactan.

The appropriate CPA is made up as a solution in water, preferably either seawater (SW) or purified water. As used herein, the term “purified water” includes deionised water, dechlorinated tap water and water purified using reverse osmosis or filtration. The term “seawater” is intended to encompass both natural and artificial seawater. The term “water” is intended to encompass both freshwater (e.g. rainwater, purified water) and seawater (natural or artificial).

In preferred embodiments of the invention, Milli-Q® water (MQ) is used, and for Pacific oyster oocytes, optimal results are obtained with MQ. The reason for the advantage of using MQ may be related to reduced osmotic stress during CPA equilibration and/or reduced exposure to high solute concentrations at any given temperature during freezing.

The CPA solution may also contain polyvinylpyrrolidone (PVP). Preferred polyvinylpyrrolidones include PVP 360, PVP 40, PVP 40T and PVP 10 (Sigma).

The bivalve oocytes, suspended in SW, are combined with the CPA solution. The oocytes and solution may be combined in several steps, with mixing between each step. Alternatively, they may be combined in a single step. It may also be desirable to wait for short periods (e.g. 10 seconds to 5 min) between each step. Optimally the final concentration of CPA in the diluted oocyte mixture (after the combining/mixing steps) will usually be about 10-15% v/v, and the final concentration of PVP, where used, will typically be about 0.85% w/v.

The diluted oocyte mixture may then be loaded into suitable freezing vessels such as straws or cryovials, and the freezing process commenced.

Optionally, the freezing process may begin with an initial hold at −3° C. to 30° C. for up to 20 min. Alternatively, the freezing may commence with no hold period. After the optional hold, the oocytes are cooled at a rate of preferably about 0.1° C. min⁻¹ to 20° C. min⁻¹ to a temperature of about −4° C. to −20° C. There is then a further optional hold period of up to 15 min, during which time the oocytes are held at −4° C. to −20° C. Optionally during this hold period, a check may be carried out to determine whether ice crystals have begun to form. If not, manual seeding may be performed at this time, for example using a cotton swab dipped in liquid nitrogen. Finally, the oocytes are subjected to a second cooling, at a rate of about 0.1° C. min⁻¹ to 2.5° C. min⁻¹ to a temperature of about −25° C. to −55° C. The freezing vessels containing the oocytes are then transferred to liquid nitrogen for storage.

When it is desirable to thaw the frozen oocytes, this can be carried out by preparing a liquid bath (using, for example, seawater or tap water) at about 0° C. to 95° C., but optimally about 25° C. and thawing the oocyte samples in this liquid bath. It is important that the temperature of the oocyte mixture in the freezing vessels should not exceed the upper limit of the normal physiological range during thawing. The oocyte mixture may then be diluted with a suitable volume of seawater (e.g. 0.1 to 100 mL). After 0 to 120 min, a further volume of seawater may be added (e.g. 0.1 to 100 mL). After a further 0 to 120 minutes, the oocytes are ready to be fertilised. Fertilisation should be carried out at normal physiological temperatures (e.g. preferably not below about 19° C. for Crassostrea gigas).

For Pacific oyster oocytes, the optimal cooling rate for the second cooling step was found to be 0.3° C. min⁻¹. Other groups have reported using a vide variety of cooling rates, but a rate of about 0.3° C. min⁻¹ is one of the slowest reported for marine organisms. During rapid cooling, intracellular ice formation occurs over a broad range of subfreezing temperatures in a diverse range of biological cells. Successful freezing must take account of both intracellular ice and solute effects and the optimal slow rate used for Pacific oyster oocytes suggests that the former may be a major contributor to cell damage, with a slow rate needed to allow water to leave the oocytes.

The results shown in Example 2 demonstrate that thawed oocytes are able to develop as normal larvae to spat. Example 2.7 investigates the long-term viability of cryopreserved oocytes, and the results show that the freezing method advantageously produces oocytes which can develop through to spat in sufficient numbers that the protocol will be of use to hatcheries with selective breeding programs. There is no long term detrimental effect of cryopreservation on subsequent development post-thawing. Indeed, mature adult oysters produced from cryopreserved oocytes are now growing on farms around New Zealand, and viable gametes have been obtained from them.

### EXAMPLES

The invention will now be further described with reference to the following examples. It is to be appreciated that the invention is not limited in any way to these examples.

### Example 1

### General Methods

### Example 1.1

### Garnete Recovery

Oocytes were obtained during the natural breeding season from sexually mature Pacific oysters reared at the Glenhaven Aquaculture Centre in Nelson, New Zealand or from commercial farms throughout New Zealand. Oocytes were recovered by physical stripping of the gonad after opening the shell. The maturity of the oocytes was assessed by visual examination. Recovered oocytes were washed into glass beakers containing 1 μm cartridge filtered seawater (SW) at ambient temperature (˜23° C.) and allowed to settle for approximately 60 min in a cold top set at 5° C. Settled oocytes were collected into 50 mL Falcon tubes (Becton Dickinson, Franklin Lakes, N.J.) and placed in a water bath at 5° C. where they were settled again for approximately a further 30 min. Depending on the experiment, oocytes from various individuals were either pooled or placed individually into 50 mL Falcon Tubes. The density of the oocytes was calculated and the pool or individual samples then diluted with SW to 2 million mL⁻¹. Oocytes were stored for up to 4 days at 5° C. before being used in experiments.

Sperm to be used for fertilisation of oocytes were also recovered by stripping of gonads. They were collected “dry”, without any addition of SW, and stored at 5° C. to minimize aging effects before use.

### Example 1.2

### Cryoprotectant (CPA) Solutions

CPAs were prepared in either SW or Milli-Q® water (MQ) at double the final concentration required during cryopreservation. Solutions contained either a permeating CPA only (dimethyl sulfoxide, Me₂SO; ethylene glycol, EG; propylene glycol, PG; methanol, METH) or a mixture of permeating and non-permeating and extracellular CPAs (trehalose, T; polyvinylpyrrolidone—PVP-40; PVP). All chemicals were sourced from Sigma (St Louis, Mo.). Solutions were stored at 5° C. and usually prepared fresh each day.

### Example 1.3

### Freezing and Thawing

Oocytes to be cryopreserved were added to 5 mL glass culture tubes (Kimble Glass Inc., Vineland, N.J.) and diluted 1:1 with CPA solutions at room temperature (˜23° C.). The CPA solutions were added to the oocyte suspension in 10 steps of equal volume each min for 10 min and the tubes were agitated after each addition. Cryopreservation straws (0.25 cc, IMV, France) were then loaded with the solution containing oocytes and CPA, sealed with PVC powder and laid flat on a rack. The total time at room temperature was 20 min.

Loaded straws were placed into Cryogenesis freezers (Cryologic Pty Ltd, Mt Waverly, Australia) and held at 0° C. for 5 min. The freezers were programmed to cool at 1° C. min⁻¹ to various hold temperatures (standard: −10° C., hold for 5 min) where ice formation in the straws had either already occurred or was seeded by touching the straws with a liquid nitrogen cooled cotton bud. Following the hold period, the freezers were then programmed to cool at various rates (standard: 1° C. min⁻¹) to −35° C. at which temperature the straws were plunged into liquid nitrogen and stored. After up to 24 h storage, the straws were thawed by placing into a water bath at 28° C. The straw contents were then diluted 1:1 with SW at ˜23° C., left for 30 min, then diluted again 1:9 with SW and left for a further 30 min before being used in fertilisation assays.

### Example 1.4

### Fertilisation Assays

After dilution, the thawed oocytes along with unfrozen oocytes from the same batch (controls) were fertilised using the miniaturised fertilisation assay described previously (S L Adams, J F Smith, R D Roberts, A R Janke, H F Kaspar, H R Tervit, P A Pugh, S C Webb, N G King, Aquaculture (2004) 242, 271). Thawed oocytes were fertilised at a sperm concentration of 10⁶ mL⁻¹. For the unfrozen oocytes, sperm concentrations of 10², 10³, 10⁴, 10⁵ and 10⁶ mL⁻¹ were used. Duplicate assays were conducted on each straw and assays included a negative control where no sperm were added to check for parthenogenetic activation. Development was stopped after cleavage (2-to-8 cells) by addition of formalin (0.5% final) and the percentage of eggs fertilised was determined.

### Example 1.5

### Larval Rearing

In the final experiment, oocytes were fertilised and reared through to spat to confirm that there was no effect of cryopreservation on subsequent development.

Approximately 2.2 million unfrozen or thawed oocytes from 8 individuals were added to 500 mL of SW and fertilised at a ratio of 100 sperm per oocyte. After a contact time of ˜12 min, the fertilised oocytes were transferred into tanks containing 150 L of SW and 1 mg L⁻¹ EDTA at 23° C. After 24 h, the tanks were drained through a 45-μm mesh and subsamples taken to determine the percentage of oocytes reaching D-larvae in each treatment.

Larvae from the three individuals that had the relatively high D-larval yields from cryopreserved oocytes were selected to ongrow through to spat. Larvae which had developed from oocytes from the same individual but which were frozen by different treatments were combined and their number standardized to that of their unfrozen controls (with or without PVP; Example 2.7) were combined. The number of D-larvae was standardized for each female so that the four rearing tanks (2×cryopreserved and 2×unfrozen) all contained the same number of larvae. The larvae were resuspended in 2.5 L tanks in a continuous exchange larval rearing system and fed a mixed diet of Chaetoceros calcitrans and Isochrisis galbana (A Janke, H Kaspar, N King, S Foster, Abstracts of the World Aquaculture Society Conference, 1st-4^{th} Mar. 2004, Hawaii, World Aquaculture Society, Louisiana State University, Baton Rouge, La., (2004), 312). Tanks were cleaned and larvae screened every second day to remove dead, abnormal or slow-growing larvae. Competent larvae were induced to metamorphose by bath application of 10⁻⁴ M epinephrine (S L Coon, D B Bonar, Journal of Experimental Marine Biology and Ecology, (1985), 94, 211) for 3 to 5 h. Spat were reared in downwellers initially and then transferred to upwellers after 6 to 8 d. The number of D-larvae in each treatment that developed to eyed larvae stage and spat was determined.

### Example 1.6

### Statistical Analyses

The fertility of the unfrozen oocytes used as pools in Examples 2.1-2.6 was analysed to confirm that there was no decline in fertility during storage before the oocytes were used in experiments. The sperm concentration that resulted in 50% fertilisation (EC50) was determined by using a mixed model smoother and a logit link function to fit smooth curves to the fertility data (M P Upsdell, The New Zealand Statistician, (1994), 29, 66). The EC50's were then analysed using Residual Maximum Likelihood Model (REML in GenStat).

In each example, the percentage of oocytes fertilised was analysed using a generalized linear mixed model (GLMM in GenStat) with binomial distribution, logit link and with pool as a random effect along with all interactions with pool. By fitting pool and its interactions as random terms, the significance of each treatment was assessed against its consistency within and across pools. Where a treatment effect differed markedly from pool to pool, the standard errors of the treatment means increased and the significance of the average effect was reduced. An exception to this was Examples 2.3 and 2.4 where preliminary analysis showed that a standard REML (in GenStat) with angular transformation and weighting by the totals was the preferred statistical method. This was performed on the combined data from the two experiments. In the Example 2.7, the percentage of fertilised oocytes developing to D-larvae from each individual was analysed using ANOVA (in GenStat) with angular transformation. Data on the percentage of D's developing to either eyed larvae or spat were analysed using a paired t-test without transformation. Values expressed in the results are the backtransformed predicted means±the averaged standard error (se) of the mean.

### Example 2

### Investigation of Optimum Cryopreservation Procedure for Crassostrea gigas Oocytes

A series of experiments was conducted (Examples 2.1-2.10) to optimize the cryopreservation procedure for Pacific oyster (Crassostrea gigas) oocytes. All CPA concentrations are the final post-dilution levels. Fertilisation results from each experiment were used to determine factors to be evaluated in subsequent experiments. Examples 2.1 to 2.3 used three pools of oocytes which were chosen from 7 available pools on the basis of post-thaw fertilisation. Each pool comprised oocytes from 4 to 8 oysters. The oocyte pools were frozen in 10% EG with 0.85% PVP in MQ using the standard holding temperature and cooling rates described previously and pools with post-thaw fertilisation above 40% were selected for use. Three new pools were selected from 11 available pools for Examples 2.4 to 2.6. Example 2.7 used oocytes from individuals and Examples 2.8 to 2.10 used oocytes from one pool of oysters only.

### Example 2.1

In this experiment, four CPAs (EG, MeSO, PG and methanol) were compared, as were three CPA concentrations (5, 10, 15% v/v for EG and PG: 10, 15, 20% v/v for Me₂SO and 2.5, 5, 10% v/v for methanol). All CPAs were prepared in MQ and contained 0.85% w/v PVP. Freezing and thawing was carried out using the standard holding temperature and freezing rates described previously.

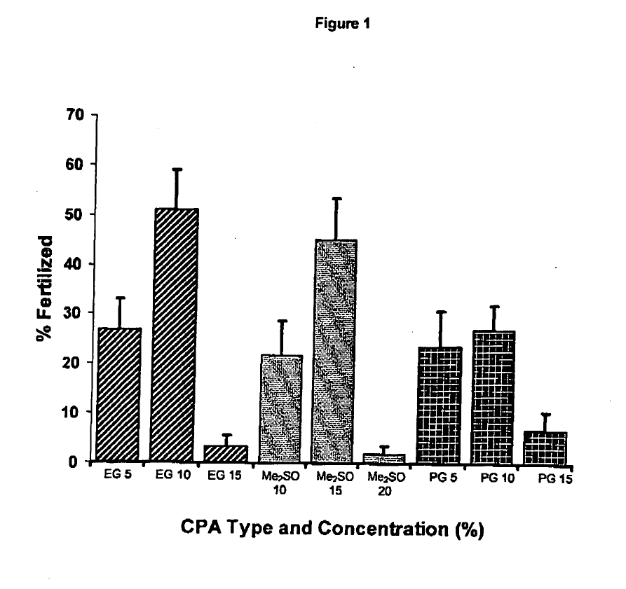

No oocytes were fertilised following cryopreservation with methanol as the CPA. Analysis of the post-thaw fertilisation data for the other CPAs showed a significant treatment effect (P<0.001; FIG. 1). Highest fertilisation rates were achieved with either 100% EG or 15% Me₂SO.

### Example 2.2

This experiment evaluated the effect of PVP and trehalose as extracellular CPAs in the presence of an effective permeating CPA (10% EG) determined from Example 2.1. Three concentrations of PVP (0.17, 0.85, 4.25%) and three concentrations of trehalose (0.1, 0.2, 0.4 M) plus a treatment containing 0.85% PVP and 0.2M trehalose were investigated. Ten percent EG in MQ was included as a control for the effect of extracellular CPA. The standard freezing/thawing protocol was used.

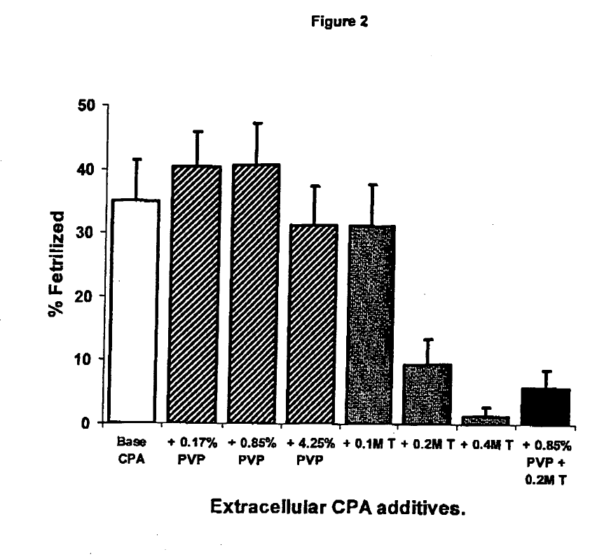

The addition of an extracellular CPA to a 10% EG base medium had a significant effect on oocyte fertilisation post-thaw (P<0.001; FIG. 2). Whereas PVP addition did not markedly affect fertilisation, the addition of trehalose above 0.1 M was associated with a significant reduction in fertilisation. The addition of both PVP and trehalose also decreased fertilisation.

### Example 2.3 and Example 2.4

A factorial design compared SW and MQ as base media and two CPAs (10% EG and 15% Me₂SO) in the presence or absence of 0.85% PVP for 3 pools of oocytes. Standard freezing/thawing protocols were again used. Example 2.4 simply duplicated Example 2.3 using three new pools of oocytes.

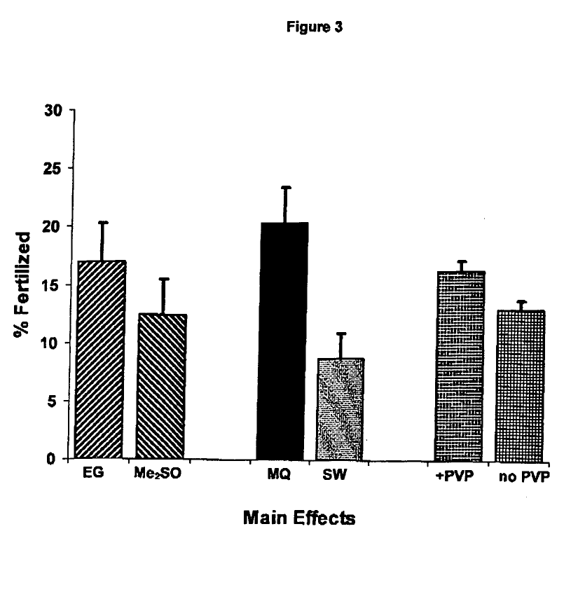

As preliminary analysis indicated no effect of experimental run, data from Examples 2.3 and 2.4 were combined into a single analysis. Fertilisation rate post-thaw was not affected by CPA (P=0.335) but significantly improved when the CPA was prepared in MQ compared to SW (P<0.001) and tended to improve when PVP was present (P=0.057). FIG. 3 illustrates these main effects.

### Example 25

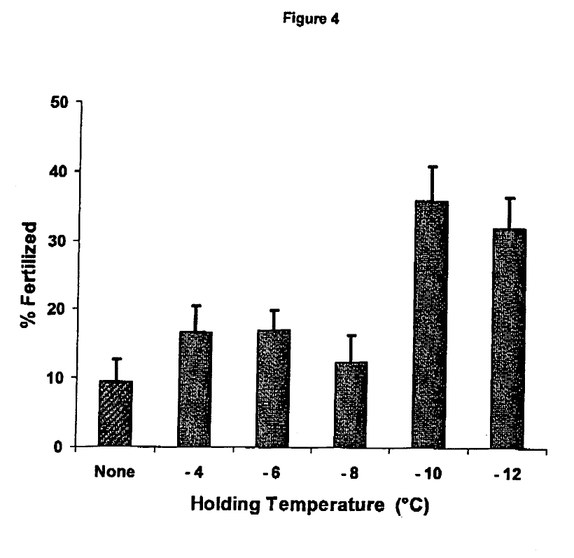

This experiment investigated the effect of holding temperature. Five holding temperatures (−4, −6, −8, −10, −12° C. for 5 min); plus a treatment involving no hold were compared. The 3 new pools of oocytes were used with 10% EG in MQ as the CPA solution. Straws were cooled at the standard rate of 1° C. min⁻¹ and on reaching the various holding temperatures, were examined for the presence of ice. Unfrozen straws were manually seeded. Following the hold, straws were cooled at the standard post-holding rate of 1° C. min⁻¹. For the treatment involving no hold, straws were cooled from 0 to −35° C. at 1° C. min⁻¹ without any examination for seeding. Oocyte fertilisation post-thaw was significantly affected by holding temperature (P<0.001, FIG. 4) with best results achieved after holding for 5 min at either −10 or −12° C.

### Example 2.6

This experiment compared four post-holding cooling rates. Straws were cooled at 1° C. min⁻¹ to −10° C., held for 5 min, and then to −35° C. at either 0.3, 1, 3 or 6° C. min⁻¹1. Ten percent EG in MQ was used as the CPA solution.

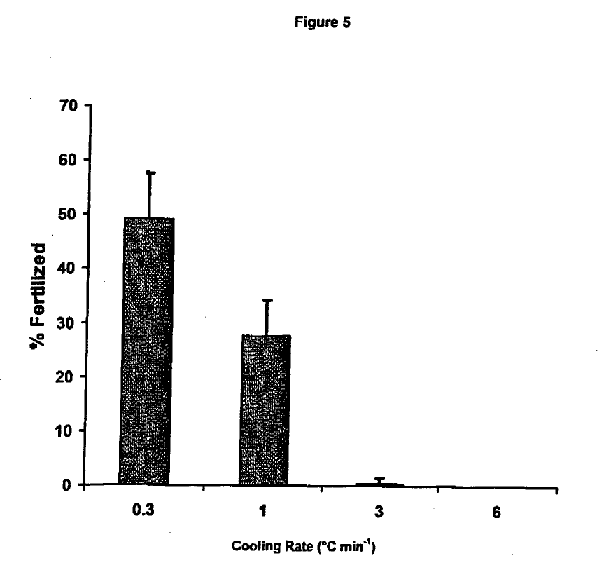

Freezing rate significantly affected oocyte fertilisation post-thaw (P<0.001) with best results achieved at the slowest rate (FIG. 5).

### Example 2.7

This experiment investigated individual female oyster variation in “freezability” and the developmental competence of cryopreserved oocytes. Oocytes from eight individuals were processed through the best treatments determined from the previous examples (10% EG in MQ with or without 0.85% PVP, cooling at 1° C. min⁻¹ to a −10° C. hold and then 0.3° C. min⁻¹ to −35° C. followed by plunging into liquid nitrogen). Ten to twelve straws from each treatment were thawed, fertilised and placed into tanks to determine development to D-larvae and beyond.

Oocyte fertilisation was improved in the absence of PVP (P=0.007; no PVP, 52.1±1.2%, PVP, 47.7±1.2%) and was affected by the individual from which the oocytes were recovered (P<0.001). Individual post-thaw fertilisation rates ranged from 2.1% to 71.7% compared to 61.0 to 97.3% for oocytes from the same individuals unfrozen. The percentage of oocytes developing to D-larvae was also low and highly variable between individuals, ranging from 2.2 to 42.3% for fresh oocytes, and 0.1 to 30.1% for cryopreserved oocytes. In general, individuals with the highest development of fresh oocytes also had high levels of development of cryopreserved oocytes. There was no significant difference in the percentage of fertilised oocytes developing to D-larvae between fresh and cryopreserved treatments (P=0.126). Likewise, there was no difference in the proportion of larvae developing to eyed larvae or spat (P=0.516 and 0.418, respectively).

### Example 2.8

This experiment investigated the effect of plunge temperature. Three plunge temperate (−25, −35 and −45° C.) were compared. Oocytes from a pool of individuals were used and 10% EG in MQ was used as the CPA solution. Straws were held at 0° C. for 5 min then cooled at 1° C. min⁻¹ to a −10° C., held for 5 min, before cooling at 0.3° C. min⁻¹ to either −25° C., −35° C. or −45° C. and then plunged into liquid nitrogen.

Plunging at −25° C. gave notably lower post-thaw fertilisation than plunging at −35° C. or −45° C.; 5% at −25° C. compared to 72 and 76% at 35° C. and 45° C., respectively.

### Example 2.9

This experiment investigated the effect of lower holding temperatures. Two holding temperatures, −16° C. and −20° C. were compared. Ten percent EG was used as the CPA solution. Straws were held at 0° C. for 5 min then cooled at 1° C. min⁻¹ to either −16° C. or −20° C., held for 5 min, then cooled at 0.3° C. min⁻¹ to −35° C. and plunged into liquid nitrogen. The post-thaw fertilisation rate of oocytes held at −16° C. was 31%. For oocytes held at −20° C., post-thaw fertilisation was poor, only 3%.

### Example 2.10

Four thawing temperatures were evaluated: 3, 28, 60 and 90° C. Straws were cryopreserved using the standard protocol (10% EG in MQ, cooling at 1° C. min⁻¹ to a −10° C. hold, held for 5 min, and then 0.3° C. min⁻¹ to −35° C. followed by plunging into liquid nitrogen) then thawed by placing into a water bath at the desired temperature until the Ice in the straw had melted. Post-thaw fertilisation was 79, 71, 61 and 52% for straws thawed in water baths at 3, 28, 60 and 90° C., respectively.

Although the invention has been described by way of example, it should be appreciated that variations and modifications may be made without departing from the scope of the invention. Furthermore, where known equivalents exist to specific features, such equivalents are incorporated as if specifically referred in this specification.

1. A method of cryopreserving bivalve shellfish oocytes, including the steps of:
(a) preparing a solution comprising 5-50% v/v of a cryoprotectant in water to give a cryoprotectant solution;
(b) combining the oocytes with the cryoprotectant solution to give an oocyte mixture;
(c) carrying out a first cooling of the oocyte mixture at a rate of about 0.1° C. min⁻¹ to 20° C. min⁻¹ to a temperature of about −4° C. to −20° C.; and
(d) carrying out a second cooling of the oocyte mixture at a rate of about 0.1° C. min⁻¹ to 2.5° C. min⁻¹ to a temperature of about −25° C. to −55° C.

2. A method as claimed in claim 1 where the cryoprotectant is a permeating cryoprotectant.

3. A method as claimed in claim 2 where the permeating cryoprotectant is selected from the group consisting of dimethyl sulfoxide, ethylene glycol, and propylene glycol.

4. A method as claimed in claim 1 where the cryoprotectant is a non-permeating cryoprotectant.

5. A method as claimed in claim 4 where the non-permeating cryoprotectant is selected from the group consisting of trehalose, sucrose, and arabinogalactan.

6. A method as claimed in claim 1 where the oocytes are suspended in or combined with seawater at a density of about 1600 to 2,000,000 oocytes mL⁻¹.

7. A method as claimed in claim 1 where the first cooling of the oocyte mixture in step (c) is carried out at a rate of about 1° C. min⁻¹.

8. A method as claimed in claim 1 where the second cooling of the oocyte mixture in step (d) is carried out at a rate of about 0.3° C. min⁻¹.

9. A method as claimed in any one of the preceding claims where the cooling of the oocyte mixture in step (c) is carried out to a temperature of about −10° C. to −16° C.

10. A method as claimed in claim 1 where the cooling of the oocyte mixture in step (d) is carried out to a temperature of about −35° C. to 45° C.

11. A method as claimed in claim 1 which includes a further step (e) between steps (b) and (c):
(e) loading the oocyte mixture into one or more vessels suitable for freezing.

12. A method as claimed in claim 1 which includes a further step (f) between steps (c) and (d):
(f) checking whether ice crystals have formed in step (c), and, if no ice crystals have formed, manually seeding the oocyte mixture to induce ice formation.

13. A method as claimed in claim 1 which includes a further step (g) between steps (c) and (d),
(g) holding the oocyte mixture at a temperature of about −4° C. to −20° C. for up to 15 minutes.

14. A method as claimed in claim 1 where the solution in step (a) comprises about 10-40% v/v of the cryoprotectant.

15. A method as claimed in claim 1 where the concentration of the cryoprotectant in the oocyte mixture after step (b) is about 10-15% v/v.

16. A method as claimed in claim 1 which includes a further step (h) between steps (b) and (c);
(h) holding the oocyte mixture at a temperature of about −3° C. to 30° C. for up to 60 minutes.

17. A method as claimed in claim 1 which includes a further step (i) after step (d):
(i) holding the oocyte mixture at a temperature of about −25° C. to −55° C. for up to about 20 minutes.

18. A method as claimed in claim 17 which includes a further step (j) after step (i) or step (d):
(j) storing the oocyte mixture at a temperature below about −130° C.

19. A method as claimed in claim 18 which includes a further step (k) after step (j):
(k) thawing the oocyte mixture at about 20° C. to 30° C.

20. A method as claim in claim 1 where the bivalve shellfish oocytes are oocytes of any one of the genera selected from the group consisting of Crassostrea, Saccostrea, Perna, Mytilus, Tapes, Ruditapes, Mercenaria, Panopea, Pinctada, Tridacna, Anadara, Ostrea, Pecten, Argopecten, and Tiostrea.

21. A method as claimed in claim 1 where the bivalve shellfish oocytes are oocytes of any one of the species selected from the group consisting of Crassostrea ariakensis, Crassostrea virginica, Crassostrea sikarnea, Saccostrea glomerata (commercialis), Saccostrea cucullata, Perna viridis, Perna canaliculus, Perna perna, Mytilus edulis, Mytilus galloprovincialis, Mytilus trossulus, Ruditapes philippinarum, Ruditapes variegata, Ruditapes decussates, Ruditapes largillierti, Mercenaria mercenaria, Pecten yessoensis, Pecten maximus, Pecten fumatus, Argopecten irradians, Pecten novaezelandiae, Panopea abrupta, Panopea zelandica, Pinctada margaritifera, Pinctada maxima, Pinctada fucata, Tridacna gigas, Tridacna maxima, Tridacna squamosa, Tridacna derasa, Anadara granos, a Ostrea edulis, Ostrea chilensis, Ostrea angasi, and Austrovenus stutchburyl.

22. A method as claimed in claim 1 where the bivalve shellfish oocytes are oocytes of Crassostrea gigas (Pacific oyster).
